SEQUENCE LISTING (3 sequences)

SEQ ID NO: 1 - DNA, 1271 bp, synthetic construct
  source 1..1271: mol_type = other DNA
gcagcagattgggatggcacgccggttccagcagatgcaggaccgggcaatacgtgggaacttcatccgcttagcgatgattttaattatagcgcacctgcatcaggaaaatcagcaacgttttttgaacgctggtcagaaggctttatcaatccgtggttaggactgggagaaacagaatattatggaccgaatagctcagttgaaagcggaaatttagttattaaagcatctcgcaaagcaggcacgacaaaaattcatgcaggcgcaattcattctaatgaaagcgttacgtatcctctgtatatggaagcacgagttcaagttacaaatctgacgatggcaaatgcattttggttactgtctagcgattctacacaagaaatcgatgttcttgaaagctatggctctgatagaccgtctgaaacgtggtttgatgaacgccttcatctgagccatcatgtttttattcgcgaaccgtttcaagattatcaacctaaagatgatggctcttggtatcctaatccgaatggaggcacgtggagagatcaatggattcgcatcggaacatattgggttgatccttggacacttgaatattatgttaatggcgaacatgttcgcacagttacgggcccttctatgatcgatccgtatggctatacgggcggaacgggactgtctaaacctatgcaagttatctttgatgcagaacatcaaccgtggcgcgatacacaaggcacagcacctccgacggatgaagaacttgcagatccgagccgcaataaatttcttgttgattgggttcgcttttataaacctgttccggatacgaatggaggagggcccggaaatggctcaatcagcgttgaaaaagaagcagaagattttgataatgttggcggctattttagcgatggccaaagccaagcaatctctacatatacgacgggcgcaacgacagcaattaattatgttaatagagaagattatgcagattatacagttacggttcctgaagatcgcatctataatatcacgtataatatctctagcggcattacaggaggccgcatcgattttcttgttaatgaatcaggcacgtggtcaaataaaacacaaacggcagttccgaatgcaggctggaataattttcaaccgctgagcggcggcacagtttatttagaagcaggaacacatacggttcgcctgtatggagcaggaacacatgattggcaatggaatctggataaatttacactgtctaataa

SEQ ID NO: 2 - DNA, 1002 bp, synthetic construct
  source 1..1002: mol_type = other DNA
caagcacaagattgggcaggcattccggtcccggcagatccgggcaatggcaaacaatggaaacttcaagcagatatgtcagatgattttaattatgattttccggcgaataaagaagaaacgtatatcgcaggcaaatggaaaaatttttggcataatagctgggatggaccgggaccgacacaatggcgccatgaaaatgtctcagtttctaatggccacatgaatattgttgcgagccgtaatggcaatacgaaaacgtttcgtaattcacatgatggaacgtatcatacgttacctgcgacacaaatgggctgcgttgtgtctaaaggacatgttcaatatcctgtgtttgtcgaagcgcgcgttaaaatcgcggatgcagtctttgcgaataatgtctggatgatctctgatgatgattatgaagaaatcgacatttgcgaaaattatggaggcttgggcgatcctggccgtacaggaacagcgatgaatgcgtggtttgcaaaacatattcatcttagccatcatgtctttaataatcgccatcttacgaattttgatgattatcaacctagagatgaagaaggagtgtatggaacgtggtattatgaaaatggcagaactgattgggcgggagaatatagcacgatcggagtgtattggaaagatcctaatcatttagaatattatatcaatggcaaatgggttcgcacgctgagcggcaaaaattatagctatttagatccggatggcaaactgatcgaagcgtcagcggattttaatgtgcttgataaatataattatacgaatggcaaaggactgacgaaacctatgaaacttatcatcaatattgaagcacaagattggaatgcacttgcaggacgctatcctacagatggagaaatctatggccgccctgaagatcatatcatgaaagtcgattggattcgggtgtatacaccggaagttgtgacgggacatcatcatcatcatcattaa

SEQ ID NO: 3 - DNA, 1125 bp, synthetic construct
  source 1..1125: mol_type = other DNA
atgagcgatagcaaagttaataaaaaactgtctaaagcaagccttagagcaatcgaacgcggctatgatgaaaaaggacctgaatggttatttgaatttgatattacacctcttaaaggagatttagcttatgaagaaggcgttattagacgcgatccgagcgcagttcttaaagttgatgatgaatatcatgtttggtatacgaaaggagaaggagaaacggttggctttggctcagataatccggaagataaagtttttccgtgggataaaacagaagtttggcacgcaacgtctaaagataaaattacgtggaaagaaatcggccctgcaattcaacgcggcgcagcaggcgcgtatgatgatcgcgcagtttttacaccggaagttcttagacataatggcacgtattatcttgtttatcaaacggttaaagcaccgtatcttaatcgctcactggaacatattgcaattgcatatagcgattctccgtttggaccgtggacgaaatcagatgcacctatcctgagccctgaaaatgatggcgtttgggatacggatgaagataatcgctttcttgttaaagaaaaaggctcatttgattcacataaagttcatgatccttgccttatgttttttaataatcgcttttatctgtattataaaggagaaacaatgggagaatctatgaatatggggggacgtgaaatcaaacatggcgttgcaattgcagattctcctctgggcccgtatacgaaatctgaatataatcctattacaaatagcggccatgaagttgcagtttggccgtataaaggaggcatggcaacaatgctgacgacggatggacctgaaaaaaatacgtgccaatgggcagaagatggcatcaattttgatataatgtctcatatcaaaggcgcaccggaagcagttggcttttttcgcccggaatcagatagcgatgatccgatctcaggcatcgaatggggactgtcacataaatatgatgcaagctggaattggaattatctgtgcttttttaaaacacgccgccaagttctggatgcaggcagctatcaacaaacgggcgattcaggagcagttcatcatcatcatcatcattaa

(19) United States
(12) Patent Application Publication
(10) Pub. No.: US 2025/0360174 A1
(43) Pub. Date: Nov. 27, 2025

(54) ENGINEERED BACTERIA, ENGINEERED BIOFILMS, AND LIVING MATERIALS BASED ON BACILLUS SUBTILIS

(71) Applicant: EAST CHINA UNIVERSITY OF SCIENCE AND TECHNOLOGY, Shanghai (CN)

(72) Inventors: Jiaofang HUANG, Shanghai (CN); Xiaojuan ZHU, Shanghai (CN); Ning JIANG, Shanghai (CN); Jianshu CHEN, Shanghai (CN)

(73) Assignee: EAST CHINA UNIVERSITY OF SCIENCE AND TECHNOLOGY, Shanghai (CN)

(21) Appl. No.: 19/078,339

(22) Filed: Mar. 13, 2025

(30) Foreign Application Priority Data
May 22, 2024 (CN) 202410636114.9

(51) Int. Cl. A61K 35/742; A61P 1/00; A61P 29/00; C12N 1/20; C12N 9/38; C12R 1/125

(52) U.S. Cl. CPC A61K 35/742; A61P 1/00; A61P 29/00; C12N 1/20; C12N 9/2468; C12R 2001/125; C12Y 302/01081; C12Y 302/01159

(57) ABSTRACT

An engineered bacterium based on Bacillus subtilis is provided, a genome of the engineered bacterium includes a multi-enzyme element, and the multi-enzyme element comprises a class I β-agarase element (E1 element), a class II β-agarase element (E2 element), and an α-neoagaro-diohydrolase element (E3 element). The engineered bacterium integrates agarase genes into the Bacillus subtilis genome, achieving cascade catalysis of agarose to produce active oligosaccharides and monosaccharides, thereby utilizing the biological activity of these active oligosaccharides and monosaccharides to treat intestinal inflammation. An engineered biofilm based on Bacillus subtilis is also provided, which includes the engineered bacterium and its secretions. Furthermore, a living material for multi-enzyme display and treatment of intestinal inflammation in mice is also provided, which includes the engineered bacterium or the engineered biofilm.

### CROSS REFERENCE TO RELATED APPLICATIONS

This application claims priority to the Chinese patent application No. 202410636114.9, filed on May 22, 2024, the entire contents of which are hereby incorporated by reference.

### SEQUENCE LISTING

The instant application contains a Sequence Listing which is submitted electronically in XML format and is hereby incorporated by reference in its entirety. The XML copy, created on Jan. 21, 2025, is named “2025 Jan. 17-Sequence list-62704-H008US00,” and is 7,279 bytes in size.

### TECHNICAL FIELD

The present disclosure relates to the technical field of molecular biology, and in particular, to engineered bacteria, engineered biofilms, and living materials based on Bacillus subtilis.

### BACKGROUND

The prevalence of inflammatory bowel disease (IBD) significantly affects human health, which often causes gastrointestinal (GI) weakness symptoms and even potentially leads to cancer. Although the exact pathogenesis of IBD is not yet clear, it is known to be associated with the disruption of the gastrointestinal epithelial barrier and ecological dysbiosis of the intestinal microbiota. Conventional treatment for symptom control relies on pharmacological interventions such as aminosalicylates, antibiotics, corticosteroids, and immunosuppressants. However, the limitations of these drugs lie in their limited efficacy and the potential severe side effects such as headaches, seizures, neurological disorders, and nephrotoxicity. Therefore, it is an urgent challenge to develop a simple, safe, and effective treatment strategy for IBD to induce effective remission and long-term maintenance.

For a long time, dietary interventions have been considered an acceptable and sustainable method for controlling symptoms of IBD by modulating the microbiota, tight junctions, and mucosal layer. However, traditional IBD dietary therapies are random, and there are significant individual differences in responses to specific foods. It is considered a promising alternative approach to transform conventional foods into more specific and active substances, which can treat IBD more precisely and effectively. Agarose is a type of natural marine polysaccharide that is widely distributed in edible red algae such as Gracilaria and Gelidium. Studies have shown that the degradation products of agarose (e.g., agar oligosaccharides) possess significant anti-inflammatory, antioxidant, and prebiotic activities. It would be a convenient and effective dietary therapy method to utilize microbes to degrade agarose into active oligosaccharides in the gastrointestinal tract for the treatment and prevention of enteritis. However, the most significant barrier to achieve this goal is that the human body typically cannot convert regular foods into specific active substances due to the lack of relevant microbes or functional enzymes in the gut.

Therefore, the probiotic Bacillus subtilis is genetically engineered to successfully display three enzymes capable of degrading agarose. The engineered Bacillus subtilis can achieve multi-enzyme cascade catalysis of agarose to produce active oligosaccharides, which is expected to effectively treat intestinal inflammation in animals.

### SUMMARY

One or more embodiments of the present disclosure provide an engineered bacterium, a genome of the engineered bacterium comprises a multi-enzyme element, the multi-enzyme element comprises a class I β-agarase element (E1 element), a class II β-agarase element (E2 element), and an α-neoagarobiose hydrolase element (E3 element).

In some embodiments, a strain name of the engineered bacterium is BS2024E1E2E3, and the engineered bacterium is deposited in the China Center for Type Culture Collection (CCTCC), with a depository number of CCTCC M 2024966.

In some embodiments, the engineered bacterium is Bacillus subtilis.

In some embodiments, the E1 element comprises a nucleotide sequence shown as SEQ ID No. 1, the E2 element comprises a nucleotide sequence shown as SEQ ID No. 2, and the E3 element comprises a nucleotide sequence shown as SEQ ID No. 3.

One or more embodiments of the present disclosure provide an engineered biofilm, which comprises the above engineered bacterium and secretions of the engineered bacterium.

One or more embodiments of the present disclosure provide a living material for multi-enzyme display and treatment of intestinal inflammation in mice, which comprises the above engineered bacterium.

One or more embodiments of the present disclosure provide a living material for multi-enzyme display and treatment of intestinal inflammation in mice, which comprises the above engineered biofilm.

### BRIEF DESCRIPTION OF THE DRAWINGS

The present disclosure will be further illustrated by way of exemplary embodiments, which will be described in detail through the accompanying drawings, wherein:

FIG. 1 is transmission electron microscopy (TEM) and immunofluorescence (IF) images of engineered biofilms according to some embodiments of the present disclosure;

FIG. 2 is a thin-layer chromatography (TLC) image of active oligosaccharides and monosaccharides produced by agarose catalyzed by different agarases displayed by engineered biofilms according to some embodiments of the present disclosure;

FIGS. 3A-3G are diagrams illustrating a therapeutic effect of engineered biofilms and agarose on intestinal inflammation in mice according to some embodiments of the present disclosure; and

FIGS. 4A-4F are diagrams illustrating evaluation results of the gastrointestinal resistance and biosafety of engineered bacterium according to some embodiments of the present disclosure.

### DETAILED DESCRIPTION

In order to provide a clearer understanding of the technical solutions of the embodiments described in the present disclosure, a brief introduction to the drawings required in the description of the embodiments is given below. It is evident that the drawings described below are merely some examples or embodiments of the present disclosure, and for those skilled in the art, the present disclosure may be applied to other similar situations without exercising creative labor, unless otherwise indicated or stated in the context, the same reference numerals in the drawings represent the same structures or operations.

It should be understood that, although the terms “first”, “second”, “third”, etc., may be used in the present disclosure to describe various elements, these elements should not be limited by these terms. These terms are used solely to distinguish one element from another. For example, a first product may be referred to as a second product, and similarly, within the scope of exemplary embodiments of the present disclosure, the second product may be referred to as the first product.

Set forth in the present disclosure and the claims, unless explicitly indicated otherwise in the context, words such as “one”, “a”, “an”, and/or “the” do not specifically denote the singular form and may also include the plural form. In general, the terms “comprising” and “including” only suggest the inclusion of steps and elements that have been explicitly identified, and these steps and elements do not constitute an exclusive listing; methods may also include other steps or elements.

Unless otherwise defined, all technical and scientific terms used in the present disclosure have the same meaning as typically understood by those of ordinary skill in the art to which the present disclosure pertains.

The following are definitions of some terms used in the present disclosure.

As used herein, “engineered bacterium” refers to new microbial cell strains or lines with specific functions obtained by modifying microorganisms through modern bioengineering techniques.

As used herein, “engineered biofilm” refers to a biofilm with specific functions, which is obtained by artificially designing and modifying microorganisms.

As used herein, “cascade catalysis” is a multi-step catalytic reaction process that typically involves multiple consecutive catalytic steps, where the product of each step serves as the substrate for the next step.

As used herein, “agarase” refers to a class of proteins capable of degrading agar (agarose), belonging to glycoside hydrolases. According to different action modes, agarases may be divided into two classes: α-agarase and β-agarase. α-agarase cleaves α-1,3 glycosidic bonds of agarose to produce a series of agarooligosaccharides (AOS) with β-D-galactose as the non-reducing end and 3,6-anhydro-α-L-galactose (L-AHG) as the reducing end; and β-agarase cleaves β-1,4 glycosidic bonds of agarose to produce a series of neoagarooligosaccharides (NAOS) with β-D-galactose as the reducing end and L-AHG as the non-reducing end.

As used herein, “agar oligosaccharides” refers to a class of oligosaccharides produced by the degradation of agarose, including AOS and NAOS. AOS can inhibit the induction of tumor necrosis factor (TNF-α) and induce the production of heme oxygenase-1, proving that AOS may be an effective method for treating inflammatory bowel disease (IBD). Further degradation of AOS and NAOS produces monosaccharide degradation products L-AHG, which has been shown to have anti-inflammatory activity in RAW264.7 cells.

As used herein, “class I β-agarase” belongs to β-agarase, which can cleave β-1,4 glycosidic bonds of agarose, and the main enzymatic products are neoagarotetraose.

As used herein, “class II β-agarase” belongs to β-agarase, which can cleave β-1,4 glycosidic bonds of agarose, and the main enzymatic products are neoagarobiose.

As used herein, “α-neoagarobiose hydrolase” belongs to α-agarase, which can cleave α-1,3 glycosidic bonds of agarose, and the main enzymatic products are L-AHG.

As used herein, “multi-enzyme display” refers to the simultaneous assembly of multiple enzymes on the cell surface or on specific carriers to achieve efficient catalytic reactions or biotransformation processes.

As used herein, “expression” includes any step involved in the production of polypeptides, including but not limited to transcription, post-transcriptional modification, translation, post-translational modification, and secretion.

As used herein, “nucleotide sequence” refers to a sequence of nucleotide from the 5′ to 3′ of a nucleic acid molecule and includes DNA or RNA molecules, including cDNA, DNA fragments or portions, genomic DNA, synthetic DNA (e.g., chemically synthesized DNA), plasmid DNA, mRNA, and antisense RNA, any of which may be single-stranded or double-stranded.

IBD significantly affects human health, which often causes gastrointestinal (GI) weakness symptoms and even leads to cancer. Probiotic therapy has shown great potential for application in this field, with its sustainability and site-specificity. However, most probiotics struggle to survive in the complex environment of the gastrointestinal tract. To address these issues, the present disclosure aims to use safe and non-toxic Bacillus subtilis to construct engineered bacterium to form an engineered biofilm. This engineered biofilm can resist the harsh environment of the gastrointestinal tract and successfully display class I β-agarase, class II-agarase, and α-neoagarobiose hydrolase, thereby producing oligosaccharides and monosaccharides with therapeutic activity against intestinal inflammation.

One or more embodiments of the present disclosure provide an engineered bacterium, whose genome includes integrated and expressed agarose degradation elements and biofilm component elements.

In some embodiments, the engineered bacterium comprises a multi-enzyme element, and the multi-enzyme element comprises a class I β-agarase element (E1 element), a class II β-agarase element (E2 element), and an α-neoagarobiose hydrolase element (E3 element). The E1 element encodes a class I β-agarase, the E2 element encodes a class II β-agarase, and the E3 element encodes an a-neoagarobiose hydrolase.

In some embodiments, the E1 element comprises a nucleotide sequence shown as SEQ ID No. 1, the E2 element comprises a nucleotide sequence shown as SEQ ID No. 2, and the E3 element comprises a nucleotide sequence shown as SEQ ID No. 3.

SEQ ID NO. 1:
GCAGCAGATTGGGATGGCACGCCGGTTCCAGCAGATGCAGGACCGGGCAATA
CGTGGGAACTTCATCCGCTTAGCGATGATTTTAATTATAGCGCACCTGCATCAG
GAAAATCAGCAACGTTTTTTGAACGCTGGTCAGAAGGCTTTATCAATCCGTGGT
TAGGACTGGGAGAAACAGAATATTATGGACCGAATAGCTCAGTTGAAAGCGGA
AATTTAGTTATTAAAGCATCTCGCAAAGCAGGCACGACAAAAATTCATGCAGGC
GCAATTCATTCTAATGAAAGCGTTACGTATCCTCTGTATATGGAAGCACGAGTTC
AAGTTACAAATCTGACGATGGCAAATGCATTTTGGTTACTGTCTAGCGATTCTAC
ACAAGAAATCGATGTTCTTGAAAGCTATGGCTCTGATAGACCGTCTGAAACGTG
GTTTGATGAACGCCTTCATCTGAGCCATCATGTTTTTATTCGCGAACCGTTTCAA
GATTATCAACCTAAAGATGATGGCTCTTGGTATCCTAATCCGAATGGAGGCACG
TGGAGAGATCAATGGATTCGCATCGGAACATATTGGGTTGATCCTTGGACACTT
GAATATTATGTTAATGGCGAACATGTTCGCACAGTTACGGGCCCTTCTATGATC
GATCCGTATGGCTATACGGGCGGAACGGGACTGTCTAAACCTATGCAAGTTAT
CTTTGATGCAGAACATCAACCGTGGCGCGATACACAAGGCACAGCACCTCCGA
CGGATGAAGAACTTGCAGATCCGAGCCGCAATAAATTTCTTGTTGATTGGGTTC
GCTTTTATAAACCTGTTCCGGATACGAATGGAGGAGGGCCCGGAAATGGCTCA
ATCAGCGTTGAAAAAGAAGCAGAAGATTTTGATAATGTTGGCGGCTATTTTAGC
GATGGCCAAAGCCAAGCAATCTCTACATATACGACGGGCGCAACGACAGCAAT
TAATTATGTTAATAGAGAAGATTATGCAGATTATACAGTTACGGTTCCTGAAGAT
CGCATCTATAATATCACGTATAATATCTCTAGCGGCATTACAGGAGGCCGCATC
GATTTTCTTGTTAATGAATCAGGCACGTGGTCAAATAAAACACAAACGGCAGTT
CCGAATGCAGGCTGGAATAATTTTCAACCGCTGAGCGGCGGCACAGTTTATTTA
GAAGCAGGAACACATACGGTTCGCCTGTATGGAGCAGGAACACATGATTGGCA
ATGGAATCTGGATAAATTTACACTGTCTAATAA
SEQ ID NO. 2:
CAAGCACAAGATTGGGCAGGCATTCCGGTCCCGGCAGATCCGGGCAATGGCA
AACAATGGAAACTTCAAGCAGATATGTCAGATGATTTTAATTATGATTTTCCGGC
GAATAAAGAAGAAACGTATATCGCAGGCAAATGGAAAAATTTTTGGCATAATAG
CTGGGATGGACCGGGACCGACACAATGGCGCCATGAAAATGTCTCAGTTTCTA
ATGGCCACATGAATATTGTTGCGAGCCGTAATGGCAATACGAAAACGTTTCGTA
ATTCACATGATGGAACGTATCATACGTTACCTGCGACACAAATGGGCTGCGTTG
TGTCTAAAGGACATGTTCAATATCCTGTGTTTGTCGAAGCGCGCGTTAAAATCG
CGGATGCAGTCTTTGCGAATAATGTCTGGATGATCTCTGATGATGATTATGAAG
AAATCGACATTTGCGAAAATTATGGAGGCTTGGGCGATCCTGGCCGTACAGGA
ACAGCGATGAATGCGTGGTTTGCAAAACATATTCATCTTAGCCATCATGTCTTTA
ATAATCGCCATCTTACGAATTTTGATGATTATCAACCTAGAGATGAAGAAGGAGT
GTATGGAACGTGGTATTATGAAAATGGCAGAACTGATTGGGCGGGAGAATATA
GCACGATCGGAGTGTATTGGAAAGATCCTAATCATTTAGAATATTATATCAATGG
CAAATGGGTTCGCACGCTGAGCGGCAAAAATTATAGCTATTTAGATCCGGATGG
CAAACTGATCGAAGCGTCAGCGGATTTTAATGTGCTTGATAAATATAATTATACG
AATGGCAAAGGACTGACGAAACCTATGAAACTTATCATCAATATTGAAGCACAA
GATTGGAATGCACTTGCAGGACGCTATCCTACAGATGGAGAAATCTATGGCCG
CCCTGAAGATCATATCATGAAAGTCGATTGGATTCGGGTGTATACACCGGAAGT
TGTGACGGGACATCATCATCATCATCATTAA
SEQ ID NO. 3:
ATGAGCGATAGCAAAGTTAATAAAAAACTGTCTAAAGCAAGCCTTAGAGCAATC
GAACGCGGCTATGATGAAAAAGGACCTGAATGGTTATTTGAATTTGATATTACA
CCTCTTAAAGGAGATTTAGCTTATGAAGAAGGCGTTATTAGACGCGATCCGAGC
GCAGTTCTTAAAGTTGATGATGAATATCATGTTTGGTATACGAAAGGAGAAGGA
GAAACGGTTGGCTTTGGCTCAGATAATCCGGAAGATAAAGTTTTTCCGTGGGAT
AAAACAGAAGTTTGGCACGCAACGTCTAAAGATAAAATTACGTGGAAAGAAATC
GGCCCTGCAATTCAACGCGGCGCAGCAGGCGCGTATGATGATCGCGCAGTTTT
TACACCGGAAGTTCTTAGACATAATGGCACGTATTATCTTGTTTATCAAACGGTT
AAAGCACCGTATCTTAATCGCTCACTGGAACATATTGCAATTGCATATAGCGATT
CTCCGTTTGGACCGTGGACGAAATCAGATGCACCTATCCTGAGCCCTGAAAAT
GATGGCGTTTGGGATACGGATGAAGATAATCGCTTTCTTGTTAAAGAAAAAGGC
TCATTTGATTCACATAAAGTTCATGATCCTTGCCTTATGTTTTTTAATAATCGCTT
TTATCTGTATTATAAAGGAGAAACAATGGGAGAATCTATGAATATGGGGGGACG
TGAAATCAAACATGGCGTTGCAATTGCAGATTCTCCTCTGGGCCCGTATACGAA
ATCTGAATATAATCCTATTACAAATAGCGGCCATGAAGTTGCAGTTTGGCCGTAT
AAAGGAGGCATGGCAACAATGCTGACGACGGATGGACCTGAAAAAAATACGTG
CCAATGGGCAGAAGATGGCATCAATTTTGATATAATGTCTCATATCAAAGGCGC
ACCGGAAGCAGTTGGCTTTTTTCGCCCGGAATCAGATAGCGATGATCCGATCTC
AGGCATCGAATGGGGACTGTCACATAAATATGATGCAAGCTGGAATTGGAATTA
TCTGTGCTTTTTTAAAACACGCCGCCAAGTTCTGGATGCAGGCAGCTATCAACA
AACGGGCGATTCAGGAGCAGTTCATCATCATCATCATCATTAA

In some embodiments, a strain name of the engineered bacterium is BS2024E1E2E3, and the engineered bacterium is deposited in the China Center for Type Culture Collection (CCTCC), with a depository number of CCTCC M 2024966.

In some embodiments, the engineered bacterium is Bacillus subtilis.

One or more embodiments of the present disclosure provide an engineered biofilm, which comprises the above engineered bacterium and secretions of the engineered bacterium.

The engineered bacteria and engineered biofilm that display multiple agarases provided by the embodiments of present disclosure can colonize and function in the intestinal tract of mice, achieving cascade catalysis of agarose to generate active oligosaccharides and monosaccharides, thereby effectively treating intestinal inflammation in mice.

One or more embodiments of the present disclosure provide a living material for multi-enzyme display and treatment of intestinal inflammation in mice, which comprises the above engineered bacterium or the above engineered biofilm.

In some embodiments, the living materials are used for the preparation of active oligosaccharides and monosaccharides.

One or more embodiments of the present disclosure also provide a use of the engineered bacterium or the engineered biofilm described above in degrading agarose to produce active oligosaccharides and monosaccharides for treatment of intestinal inflammation.

At least the following technical effects are achieved by the embodiments of the present disclosure: by integrating multi-enzyme elements for degrading agarose to produce active oligosaccharides and monosaccharides into Bacillus subtilis, the engineered bacterium, the engineered biofilm, and the living material that can degrade agarose to produce active oligosaccharides and monosaccharides are developed. The engineered bacterium, the engineered biofilm, and the living material are capable of withstanding the complex environment of the gastrointestinal tract and can be used to treat intestinal inflammation, providing a simple, effective, and continuous dietary therapy method for intestinal inflammation.

### EXAMPLES

### Example 1 Construction of a Multi-Component Agarase-Displaying Bacillus subtilis Engineered Strain BS2024E1E2E3

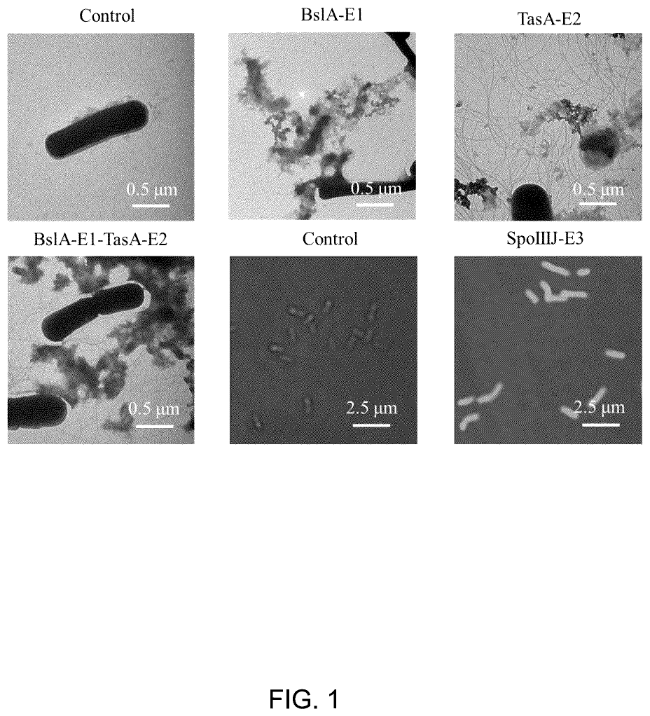

To verify the successful display of agarase on the biofilm by the engineered strain, the formation of the hydrophobic protein BslA-E1 and the TasA-E2 fiber in the engineered strain was observed using transmission electron microscopy (TEM). As shown in FIG. 1, no significant formation of the hydrophobic protein and the fiber is observed in the biofilm-defective strain negative control (Control), whereas a large amount of hydrophobic proteins and fibers form around the engineered strain that successfully expresses the BslA-E1 and TasA-E2 fusion proteins. Immunofluorescence techniques reveals that the engineered strain expressing the SpoIIIJ-E3 fusion protein exhibits significant fluorescence, while the control strain shows no significant fluorescence. Therefore, it is proven that the engineered biofilm successfully displays and expresses three agarases E1, E2, and E3.

### Example 2 Degradation of Agarose by Engineered Strains to Produce Active Oligosaccharides and Monosaccharides

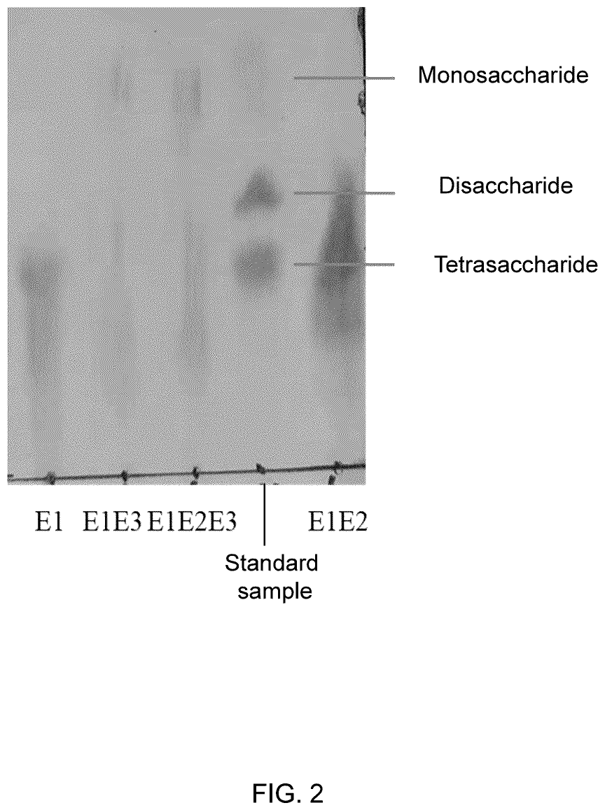

To verify the successful display of agarases in multiple components of the engineered biofilm and achieve cascade catalysis of agarose to produce active oligosaccharides and monosaccharides, engineered strains displaying individual enzyme (E1), two enzymes (E1E2, E1E3), and three enzymes (E1E2E3) were statically cultured in Minimal Spizizen's Glucose-Glycerol (MSgg) medium containing 0.1% low-melting agarose. Samples were taken every 12 hours, and the content of reducing sugars was detected using 3,5-dinitrosalicylic acid (DNS) colorimetric method. After 60 hours of enzymatic hydrolysis, samples were taken for thin-layer chromatography (TLC) to investigate the degree of polymerization of the enzymatic hydrolysis products. As shown in FIG. 2, with continuous formation of the biofilm, the enzymatic hydrolysis products gradually emerge, and the content of reducing sugars also continuously increase. TLC data shows that the enzymatic hydrolysis products of BslA-E1 are mainly neoagarotetraose, and TasA-E2 forms a cascade catalysis with BslA-E1 to enzymatically hydrolyze low-melting agarose into neoagarobiose; SpoIIIJ-E3 further enzymatically hydrolyzes the degradation products of BslA-E1 and TasA-E2 into 3,6-anhydro-L-galactose. The above experimental results indicate that E1 displayed on Bacillus subtilis expressing BslA protein and E2 displayed on Bacillus subtilis expressing TasA protein both have the ability to catalyze the degradation of agarose to produce active oligosaccharides, while E3 displayed on the cell surface can further degrade the enzymatic hydrolysis products of E1 and E2 (neoagarotetraose and neoagarobiose) into monosaccharides. This demonstrates that the rationally designed engineered Bacillus subtilis biofilm material can be used for multi-enzyme display, showing the potential for multi-enzyme synergy and cascade catalysis applications.

### Example 3 Treatment of Intestinal Inflammation by Active Oligosaccharides and Monosaccharides Produced from Agarose Catalyzed by Engineered Strains

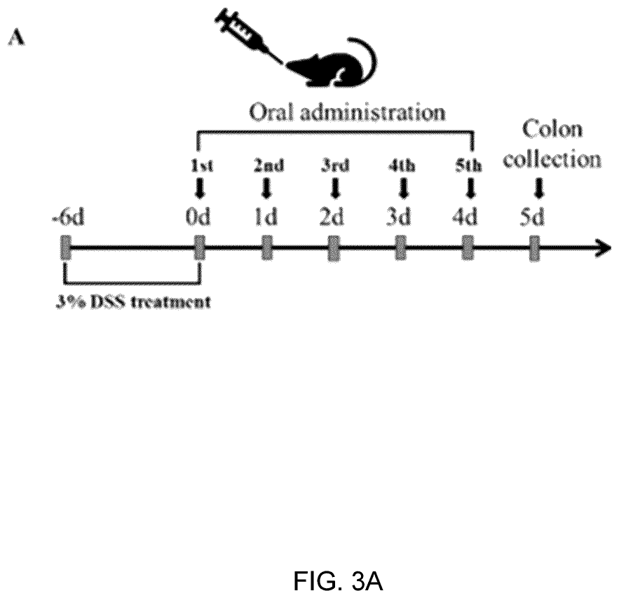

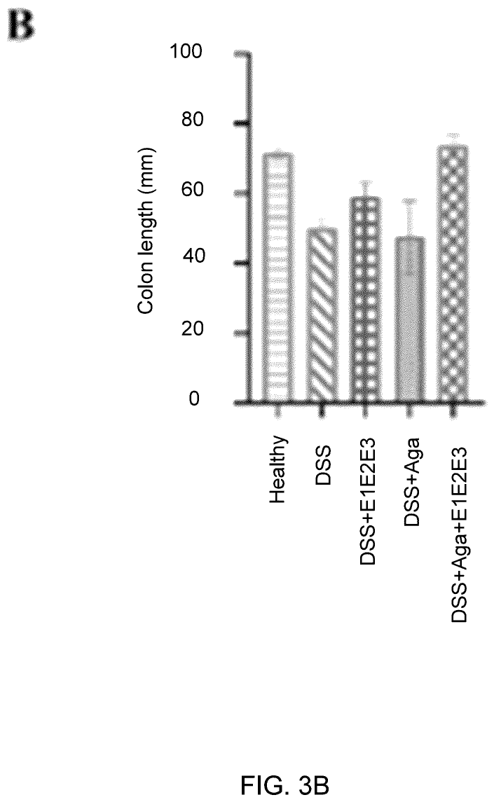

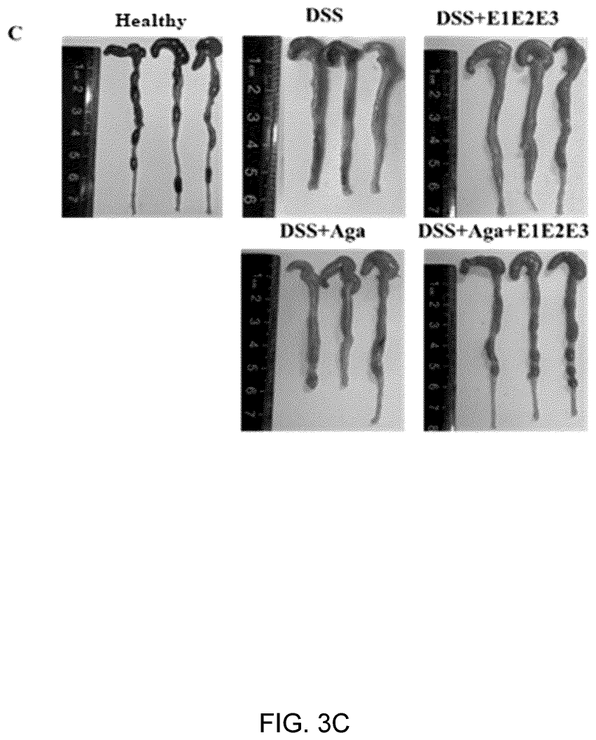

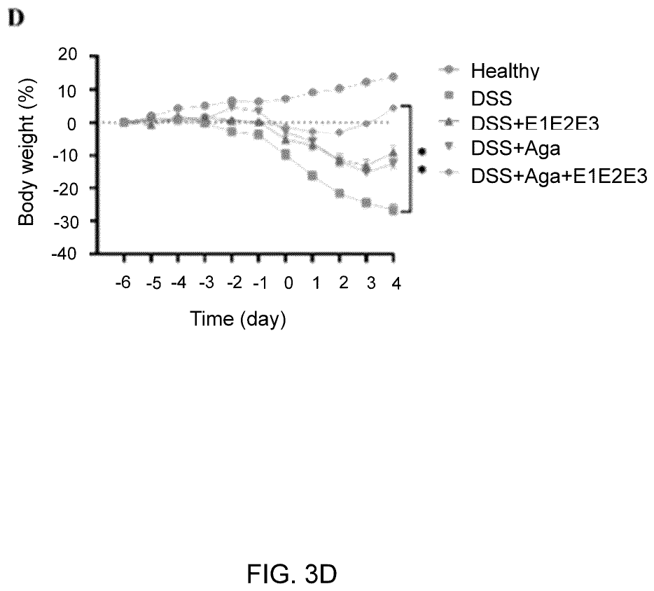

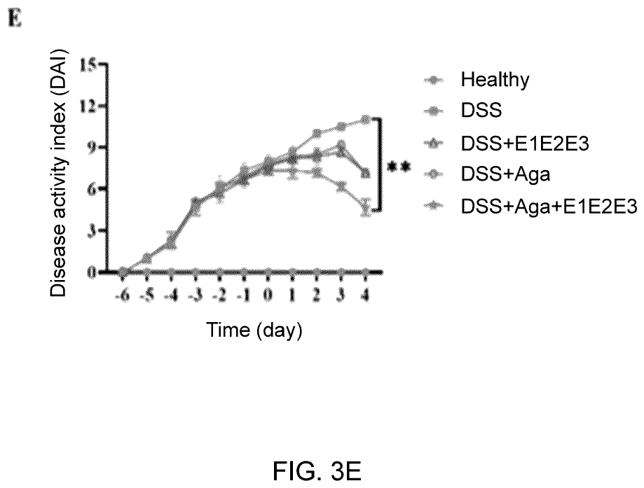

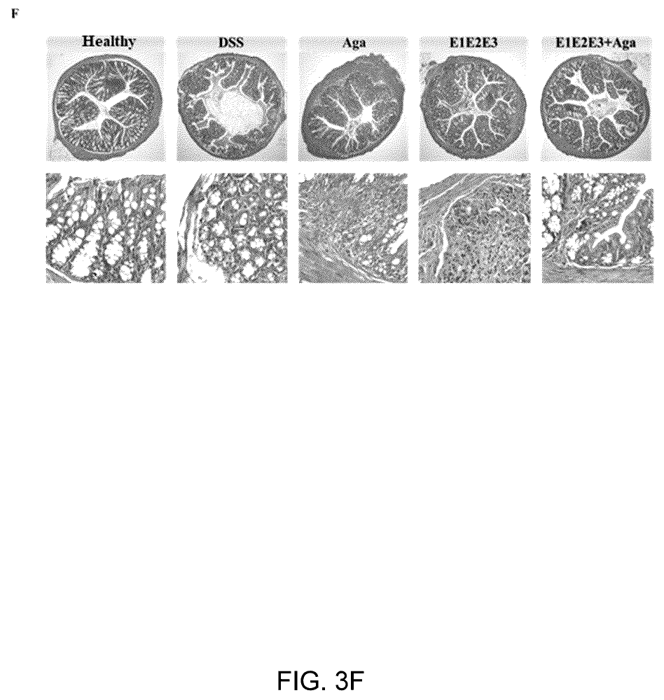

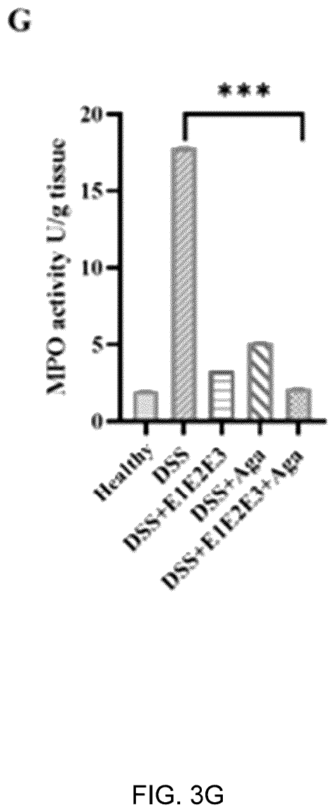

Female C57BL/6 mice, 7 weeks old, were randomly divided into five groups, with three mice in each group. The first group served as the healthy control (Healthy) and was given sterile water orally daily without any other treatment. The remaining four groups were induced to develop colitis models by freely drinking water containing 3% dextran sulfate sodium (DSS) for 5 to 7 days, during which the mice's body weight, fecal shape, and bloody stools were measured and recorded daily and scored accordingly. Once the colitis models were successfully established, DSS was discontinued and replaced with sterile water. The second group was the DSS group (DSS), which was only given phosphate-buffered saline (PBS) by oral administration; the third group was the DSS+E1E2E3 group (DSS+E1E2E3), which was given spores (1×10⁸ CFUs) of the engineered strain BS2024E1E2E3 by oral administration on the first day of treatment, followed by PBS for the next four days; the fourth group was the DSS+Aga group (DSS+Aga), which was given low-melting agarose (1×10−5 mg/kg) by oral administration daily; the fifth group was the DSS+Aga+E1E2E3 group (DSS+Aga+E1E2E3), which was given spores (1×10⁸ CFUs) of the engineered strain BS2024E1E2E3 and low-melting agarose (1×10−5 mg/kg) by oral administration on the first day of treatment, followed by low-melting agarose (1×10−5 mg/kg) for the next four days. After the fifth day of treatment, feces of the mice were collected, and then the mice were euthanized, as shown in FIG. 3A. The mice were dissected, the colon was collected, and the length and morphology of the colon were measured, as shown in FIGS. 3B and 3C. After measurement, about 1 cm of the colon was immersed in 4% paraformaldehyde for HE staining. The remaining colon was collected in sterile centrifuge tubes and stored at −80° C. for subsequent enzyme-linked immunosorbent assay (ELISA) analysis to detect colitis-related indicators.

As shown in FIGS. 3D and 3E, after induction with 3% DSS, the disease activity index of mice in the DSS group increases, exhibiting symptoms such as reduced activity, loose stools, bloody stools, and 26% of decrease in body weight. As shown in the anatomical results in FIG. 3C, compared to the healthy mice in the healthy control group, the colon of mice in the DSS group is significantly shortened, showing a significant difference. As shown in FIG. 3F, HE staining of the colon tissue reveals obvious colonic ulcers, mucosal edema, loss of goblet cells, crypt swelling and destruction, and varying degrees of inflammatory cell infiltration and epithelial cell damage in the mucosa and submucosa. As shown in FIG. 3G, by detecting the myeloperoxidase activity in the colon tissue, it is found that the myeloperoxidase activity in the colon tissue of mice in the DSS group is significantly increased. All these experimental results indicate that the colitis models in the mice are successfully established. After administering only the engineered strain BS2024E1E2E3 or 0.1% low-melting agarose, the body weight of mice recovers to some extent, but is still lower than that of healthy mice in the healthy control group; the colon length is higher than that of mice in the DSS group but lower than that of healthy mice in the healthy control group; HE staining of the colon tissue shows that colitis is not significantly alleviated, and the histopathological score is high; and myeloperoxidase activity, inflammatory factors, and other indicators are still higher than those of healthy mice in the healthy control group. However, in the DSS+Aga+E1E2E3 group administered with both the engineered strain BS2024E1E2E3 and low-melting agarose, the body weight of mice significantly recovers, and the activity increases; the colon length recovers to the level of healthy mice, and HE staining of the colon tissue shows a lower histopathological score, close to that of the healthy control group; and the epithelium is intact, the crypt structure is regular, and the percentage of inflammatory cells is low. This experiment demonstrates that simultaneously administrating the engineered strain BS2024E1E2E3 and low-melting agarose can significantly promote the repair of colon tissue of mice and effectively treat intestinal inflammation in mice.

### Example 4 Evaluation of the Biosafety of the Engineered Strain BS2024E1E2E3

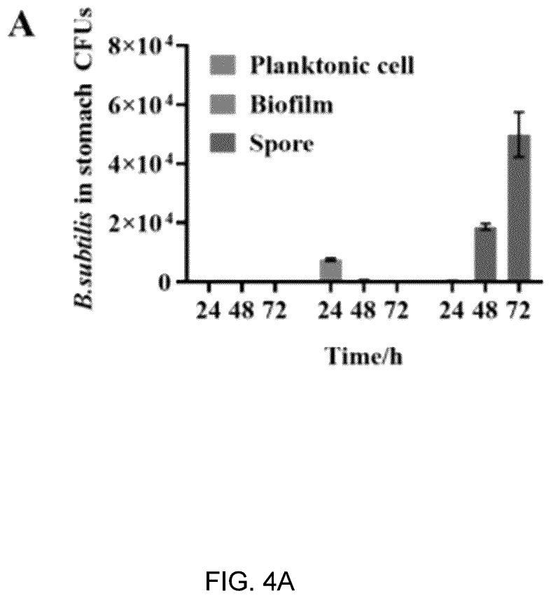

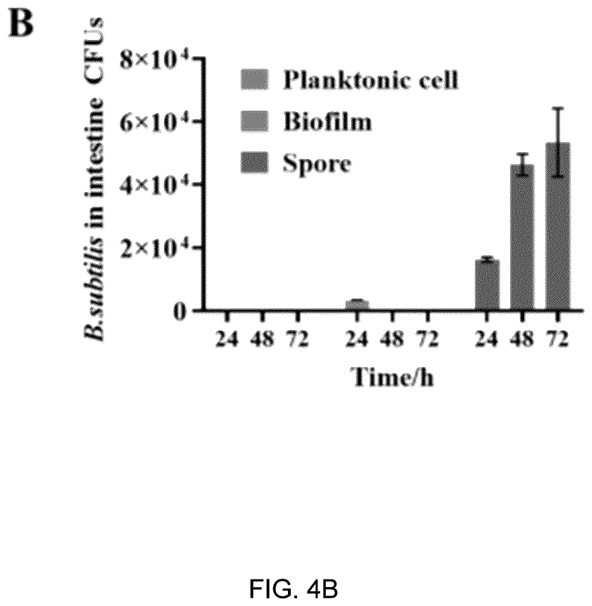

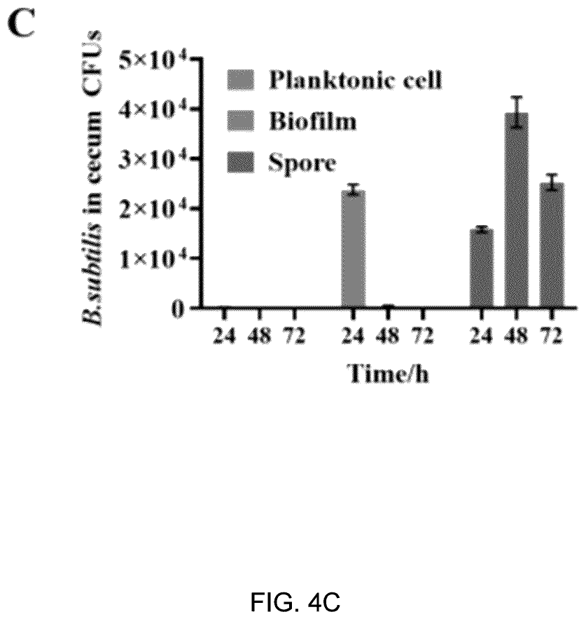

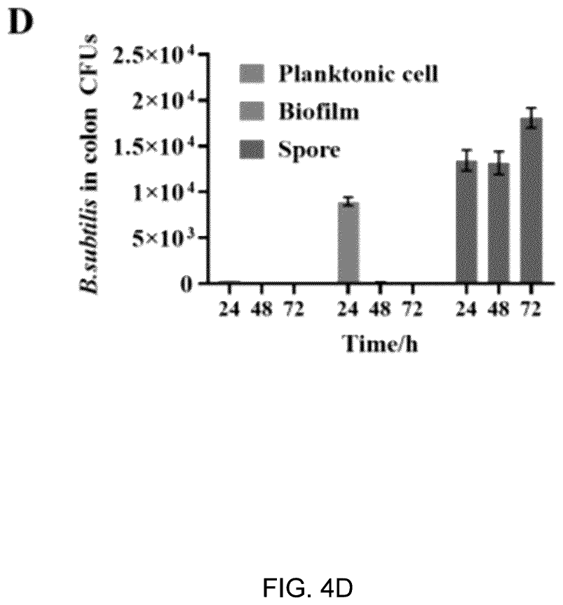

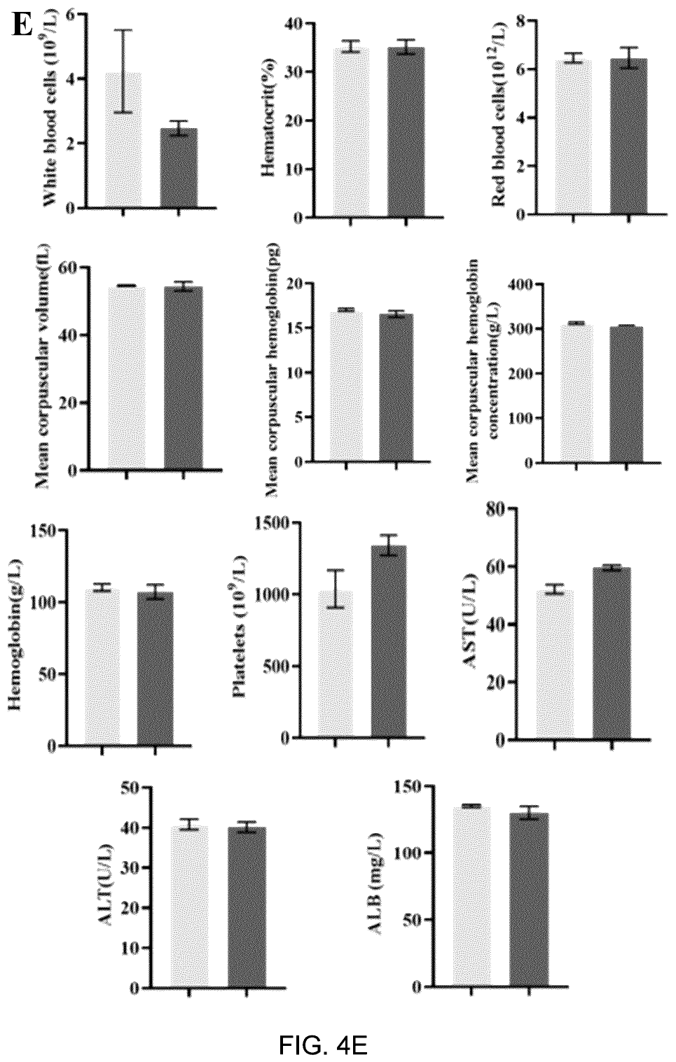

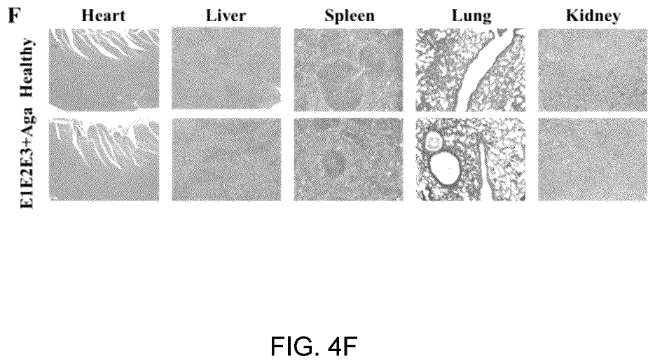

The resistance of the engineered strain to the complex environment of the gastrointestinal tract is crucial for its functional performance. To evaluate the resistance of the planktonic cells, biofilms, and spores of engineered strain BS2024E1E2E3 in the digestive tract (including the stomach, small intestine, cecum, and colon), mice were orally administered and then euthanized after 24 hours, 48 hours, and 72 hours. Subsequently, the number of engineered strain BS2024E1E2E3 in these digestive tract sites was detected. As shown in of FIGS. 4A-4D, it is indicated that the planktonic cells and biofilms of the engineered strain BS2024E1E2E3 only exhibit limited resistance to the attack of the digestive tract, while the spores of the engineered strain BS2024E1E2E3 are almost unaffected by the complex gastrointestinal environment, and about 10⁴ CFUs of the spores could still be detected in the stomach, small intestine, cecum, and colon after oral administration for 72 hours.

Healthy C57 BL/6 mice were randomly divided into two groups, namely the healthy control group (Healthy control) and the experimental group (E1E2E3+Aga). The healthy control group was healthy mice without any treatment; and the experimental group was mice orally administered with the engineered strain BS2024E1E2E3 and 0.1% low-melting agarose. On the fifth day after treatment, mice in both the healthy control group and the experimental group were euthanized, and then the main organs (heart, liver, spleen, lungs, and kidneys) and blood samples of the mice were collected. HE staining technology was used to analyze the histological changes in the organs of the mice, and a hematology analyzer (Sysmex XE-2100, Kobe, Japan) was used to test the whole blood samples of the mice. The blood parameters tested include white blood cells (WBC), red blood cells (RBC), and hemoglobin (HGB), etc. At the same time, the liver function indicators (alanine aminotransferase (ALT) and aspartate aminotransferase (AST)) and kidney function indicators (blood urea nitrogen (BUN) levels) of the mice were measured using an automatic biochemical analyzer BS-2000 m.

As shown in FIG. 4E, after multiple oral administrations of the engineered strain BS2024E1E2E3 and 0.1% low-melting agarose to mice, serum biochemical analysis and complete blood tests were performed. As expected, after treatment with the engineered strain BS2024E1E2E3 of Bacillus subtilis and 0.1% low-melting agarose, the blood cell parameters of mice, including white blood cells, red blood cells, hematocrit, mean corpuscular volume, mean corpuscular hemoglobin content, hemoglobin, and platelets, are similar to those of healthy mice. Liver function parameters and kidney function parameters, including ALT, AST, and albumin (ALB), are also within the normal range. In addition, compared to healthy mice, there is almost no difference in the body weight of the mice and tissue sections of main organs (heart, liver, spleen, lungs, and kidneys) of the mice treated with the engineered strain BS2024E1E2E3 and 0.1% low-melting agarose (shown in FIG. 4F). All these results indicate that oral administration of the engineered strain BS2024E1E2E3 of and 0.1% low-melting agarose does not cause any adverse side effects in mice.

The example of the present disclosure realizes the cascade catalytic degradation of agarose to produce active oligosaccharides by displaying agarase on multicomponent of the Bacillus subtilis biofilm. When the engineered strain and agarose are orally administered to mice with colitis induced by DSS, the engineered strain can effectively improve the colitis symptoms in mice by degrading agarose to produce the active oligosaccharides and monosaccharides, and this treatment method does not show any detectable toxic side effects, which opens up a completely new possibility in the field of dietary therapy.

The basic concepts have been described above, apparently, in detail, as will be described above, and do not constitute limitations of the disclosure. Although there is no clear explanation here, those skilled in the art may make various modifications, improvements, and corrections of the present disclosure. This type of modifications, improvements, and corrections are recommended in the present disclosure, so the modifications, improvements, and the corrections remain in the spirit and scope of the exemplary embodiment of the present disclosure.

At the same time, the present disclosure uses specific words to describe the embodiments of the present disclosure. As “one embodiment”, “an embodiment”, and/or “some embodiments” means a certain feature, structure, or characteristic of at least one embodiment of the present disclosure. Therefore, it is emphasized and should be appreciated that two or more references to “an embodiment” or “one embodiment” or “an alternative embodiment” in various parts of the present disclosure are not necessarily all referring to the same embodiment. Further, certain features, structures, or features of one or more embodiments of the present disclosure may be combined.

In addition, unless clearly stated in the claims, the order of processing elements and sequences, the use of numbers and letters, or the use of other names in the present disclosure are not used to limit the order of the procedures and methods of the present disclosure. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the subject matter of the present disclosure object requires more features than the features mentioned in the claims. Rather, the claimed subject matter may lie in less than all features of a single foregoing disclosed embodiment.

In some embodiments, the numbers expressing quantities of ingredients, properties, and so forth, used to describe and claim certain embodiments of the application are to be understood as being modified in some instances by the terms “about”, “approximate”, or “substantially”. Unless otherwise stated, “about”, “approximate”, or “substantially” may indicate ±20% variation of the value it describes. Accordingly, in some embodiments, the numerical parameters used in the disclosure and claims are approximate values, and the approximation may change according to the characteristics required by the individual embodiments. In some embodiments, the numerical parameter should consider the prescribed effective digits and adopt a general digit retention method. Although in some embodiments, the numerical fields and parameters used to confirm the breadth of its range are approximate values, in specific embodiments, such numerical values are set as accurately as possible within the feasible range.

With respect to each patent, patent application, patent application disclosure, and other material cited in the present disclosure, such as articles, books, manuals, publications, documents, etc., the entire contents thereof are hereby incorporated by reference into the present disclosure. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those described in the present disclosure, the descriptions, definitions, and/or use of terms in the present disclosure shall prevail.

At last, it should be understood that the embodiments described in the present disclosure are merely illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. An engineered bacterium, wherein a genome of the engineered bacterium comprises a multi-enzyme element, the multi-enzyme element comprises a class I β-agarase element (E1 element), a class II β-agarase element (E2 element), and an α-neoagarobiose hydrolase element (E3 element).

2. The engineered bacterium according to claim 1, wherein a strain name of the engineered bacterium is BS2024E1E2E3, and the engineered bacterium is deposited in the China Center for Type Culture Collection (CCTCC), with a depository number of CCTCC M 2024966.

3. The engineered bacterium according to claim 1, wherein the engineered bacterium is Bacillus subtilis.

4. The engineered bacterium according to claim 1, wherein the E1 element comprises a nucleotide sequence shown as SEQ ID No. 1, the E2 element comprises a nucleotide sequence shown as SEQ ID No. 2, and the E3 element comprises a nucleotide sequence shown as SEQ ID No. 3.

5. An engineered biofilm, comprising the engineered bacterium of claim 1 and secretions of the engineered bacterium.

6. A living material for multi-enzyme display and treatment of intestinal inflammation in mice, comprising the engineered bacterium of claim 1.

7. A living material for multi-enzyme display and treatment of intestinal inflammation in mice, comprising the engineered biofilm of claim 5.
